(19) United States
(12) Patent Application Publication
(10) Pub. No.: US 2026/0308121 A1
(43) Pub. Date: Oct. 8, 2026

(54) RECRYSTALLISATION OF 5-METHOXY-N,N-DIMETHYLTRYPTAMINE (5-MEO-DMT)

(71) Applicant: GH Research Ireland Limited, Dublin (IE)

(72) Inventors: Leonard Gerard Francis Patrick, Northumberland (GB); Shaun Neil Robinson, Northumberland (GB); Theis Terwey, Berlin (DE)

(73) Assignee: GH Research Ireland Limited, Dublin (IE)

(21) Appl. No.: 19/712,663

(22) Filed: Jun. 18, 2026

Related U.S. Application Data
(63) Continuation of application No. 19/564,993, filed on Mar. 12, 2026.
Application No. 19/564,993 is a continuation of application No. 17/620,854, filed on Jun. 19, 2020, now Pat. No. 12,612,363.

(30) Foreign Application Priority Data
Jun. 19, 2019 (EP) 19181211.4

(51) Int. Cl. C07D 209/16

(52) U.S. Cl. CPC C07D 209/16; C07B 2200/13

(57) ABSTRACT

A method of purifying 5-methoxy-N,N-dimethyltryptamine (5-MeO-DMT) is provided which comprises dissolving crude 5-MeO-DMT in a solvent at a temperature above room temperature, cooling the obtained solution to a temperature below room temperature to precipitate solid 5-MeO-DMT, separating the solid 5-MeO-DMT from the remaining solution and removing solvent from the crystalline 5-MeO-DMT. In the method, the solvent comprises one or more ethers and less than 5 wt % anti-solvent.

The present application is a continuation of U.S. application Ser. No. 19/564,993, filed Mar. 12, 2026, which is a continuation of U.S. application Ser. No. 17/620,854, filed Dec. 20, 2021, now U.S. Pat. No. 12,612,363, which is a U.S. National Stage of International Patent Application No. PCT/EP2020/067113, filed Jun. 19, 2020, which claims priority to European Application No. 19181211.4, filed Jun. 19, 2019. The disclosures of each of the applications listed above are incorporated by reference herein in their entireties.

### TECHNICAL FIELD

The present invention relates to a method of purifying an organic compound, specifically 5-Methoxy-N,N-dimethyltryptamine (5-MeO-DMT). The invention furthermore relates to 5-MeO-DMT in a form meeting specific purity requirements.

### BACKGROUND

5-Methoxy-N,N-dimethyltryptamine (5-MeO-DMT) has the formula shown below.

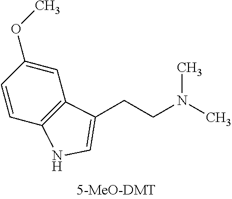 [SMILES: COc1ccc2[nH]cc(CCN(C)C)c2c1]

5-MeO-DMT is a naturally occurring serotonergic psychedelic tryptamine which acts as a 5-HT1A and 5-HT2A receptor agonist.

5-MeO-DMT is synthesized in human pineal and retina, and it has been found in human body fluids including urine, blood, and cerebrospinal fluid.

5-MeO-DMT was first isolated from the bark of Dictyoloma incanescens, but it is also contained in other plants, and it has been identified as the major active ingredient in the venom of Bufo alvarius toads.

The chemical synthesis of 5-MeO-DMT has been described in 1936 by Hoshino and Shimodaira (Bulletin of the Chemical Society of Japan, 11 (3), 221-224). No medical or other use is contemplated in this publication.

According to Hoshino and Shimodaira, following a reaction between 5-methoxy indolyl 3-ethyl beta bromide and dimethyl amine the product is isolated and purified by distillation under reduced pressure. It is also reported that the substance crystallises from ether-petrol ether. No conditions are disclosed.

The product obtained is described as nice colorless prisms having a melting point of 66-67° C. There is no characterisation regarding the amounts of impurities contained in the product.

A comparison of the reported melting point with later data regarding the melting point of 5-MeO-DMT (69-70° C.) may, however, be taken as an indication that impurities are still present.

Moreover, given the high boiling point of 5-MeO-DMT even under reduced pressure (208 to 210° C. at 4 mm), distillation is not an advantageous purification method.

Somei et al. (Chem. Pharm. Bull. 49 (1) 87-96 (2001)) report syntheses of serotonin, N-methylserotonin, bufotenine, 5-methoxy-N-methyltryptamine, bufobutanoic acid, N-(indol-3-yl)methyl-5-methoxy-N-methyltryptamine, and lespedamine.

In the context of a synthesis for bufotenine, a mixture of compounds comprising 5-MeO-DMT is obtained from which the components are purified by column chromatography. 5-MeO-DMT is then recrystallised from Et₂O-hexane. Details regarding the recrystallisation conditions or the amounts of impurities contained in the product are not disclosed. The liquid mixture used for recrystallisation (Et₂O-hexane) is similar to the mixture used by Hoshino and Shimodaira (ether-petrol ether).

Based on its physiological activities, there has recently been an interest in potential medical uses of 5-MeO-DMT, for instance, investigating potential medical uses in human clinical trials.

For such uses in human clinical trials, and for potential use in an approved medical product, 5-MeO-DMT in high purity is required. For administration to humans, purity as high as possible is necessary. According to the invention, it is in particular desirable that the total amount of impurities in the drug substance is below 0.5% and that the amount of each individual impurity is below 0.1%.

Furthermore, limits as regards the amount of residual solvent are to be observed.

Against this background, there is a need for providing a simple method of purifying 5-MeO-DMT, in particular of purifying 5-MeO-DMT so as to obtain the substance in a pharmaceutical grade. There is also a need for providing a 5-MeO-DMT in a form meeting specific purity requirements.

### SUMMARY OF THE INVENTION

The present invention relates to a method of purifying 5-methoxy-N,N-dimethyltryptamine (5-MeO-DMT). The method comprises
dissolving crude 5-MeO-DMT in a solvent at a temperature above room temperature;
cooling the obtained solution to a temperature below room temperature to precipitate 5-MeO-DMT; solid
separating the solid 5-MeO-DMT from the remaining solution;
removing solvent from the crystalline 5-MeO-DMT;
wherein the solvent comprises one or more ethers and less than 5 wt % of aliphatic hydrocarbons as anti-solvents.

The invention further provides a method comprising the steps as above, wherein the solvent comprises less than 5 wt % in total of any anti-solvents, wherein anti-solvent means a liquid in which the solubility of 5-MeO-DMT at any temperature occurring during the recrystallisation is less than 20% the solubility in the ether used, or, in case that more than one ether is used, in the combination of ethers present in the dissolution solvent.

The invention also provides 5-MeO-DMT in a form containing a total amount of impurities of below 0.5 area %, wherein the amount of each individual impurity is below 0.1 area %. The amounts of impurities are determined by chromatography as describes in detail below.

Specific embodiments are defined in the dependent claims.

### BRIEF DESCRIPTION OF THE DRAWINGS

FIG. 1 shows an example chromatogram for a 5-MeO-DMT sample analysed by HPLC as described below.

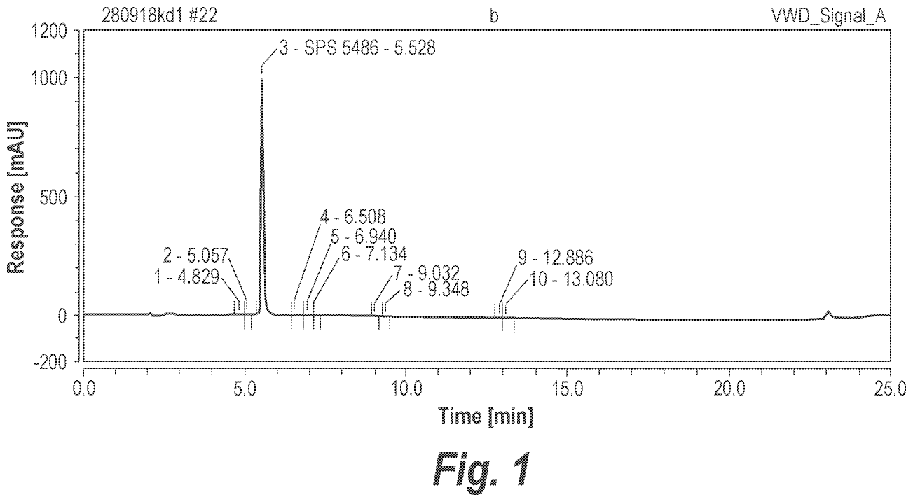

FIG. 2 shows an enhanced view of the example chromatogram.

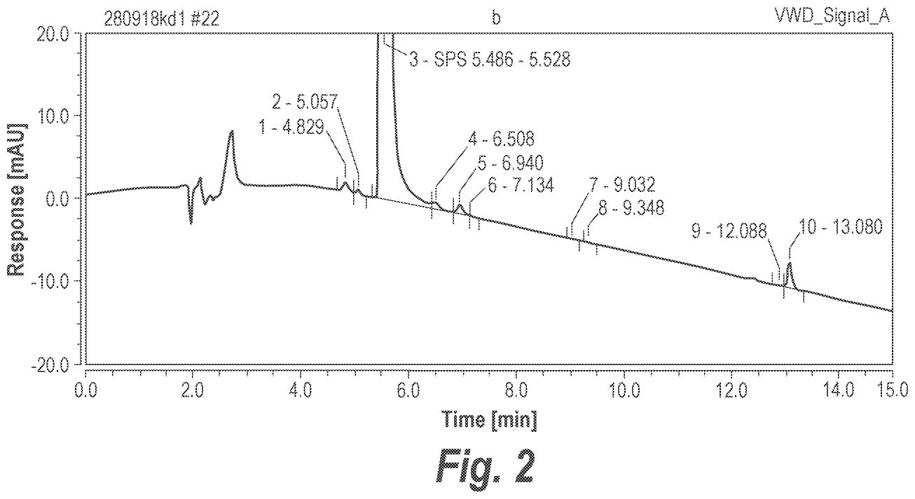

FIG. 3 is a diagram obtained by plotting the peak area, determined by HPLC, vs the concentration of 5-MeO-DMT in the analysed sample.

### DETAILED DESCRIPTION OF THE INVENTION

The present invention is based on the discovery that 5-methoxy-N,N-dimethyltryptamine (5-MeO-DMT) can be purified to obtain the substance in a pharmaceutical grade by recrystallisation if specific conditions are observed.

5-MeO-DMT is available from natural sources as well as via synthetic methods. It is a white to off white, yellow or orange powder.

The preparation of 5-MeO-DMT by isolation from natural sources or by chemical synthesis as a rule yields a material (crude 5-MeO-DMT) still containing significant amounts of impurities so that the material is not suitable for pharmaceutical applications.

The crude 5-MeO-DMT will typically contain a total amount of impurities of 0.5% or more and may contain individual impurities in amounts of 0.1% or more.

Preferably, the crude 5-MeO-DMT used in the method of the invention does not contain more than 5% impurities in total, more preferably not more than 2% impurities in total.

While colouration of 5-MeO-DMT shows the presence of one or more impurities, it does not as such indicate whether or not a certain preparation meets the requirements for a pharmaceutical use.

The purity of a 5-MeO-DMT sample is analysed by HPLC. Individual impurities can be identified by their relative retention times (RRT). The detection method is UV detection at 227 nm.

A suitable column is a reversed-phase column, in particular an octadecyl carbon chain-bonded silica (C18) column.

For elution, a mixed solvent is used which is based on a mobile phase A (0.013M ammonium acetate in water) and a mobile phase B (acetonitrile). A specific gradient method is detailed in Example 1 below.

Percent values referring to purity are area %, based on the peak area in an HPLC chromatogram obtained as described above.

The present inventors have recognised that recrystallisation of crude 5-MeO-DMT in the form of the free base, despite its relatively low melting point, is a suitable approach to obtain 5-MeO-DMT in high purity, i.e., in a form having a purity such that the total amount of impurities is below 0.5% and the amount of each individual impurity is below 0.1%.

While 5-MeO-DMT may separate from a solution upon cooling in the form of an oil, the inventors have found that solvents comprising certain ethers can suitably be used to obtain a solid, in particular crystalline, form of 5-MeO-DMT having improved purity.

The recrystallisation solvent in particular comprises one or more ethers having a boiling point in the range of 40° C. to 100° C. under a pressure of 1 bar.

Suitable ethers are aliphatic and alicyclic ethers. These ethers in particular do not comprise any functional group besides the ether moiety.

Examples of suitable ethers (boiling points in brackets) include di-n-propylether (90° C.), di-iso-propylether (69° C.), methyl tert.-butyl ether (MTBE) (55° C.), ethyl tert.-butyl ether (ETBE) (73° C.), tert.-amyl methyl ether (TAME; 2-methoxy-2-methylbutan) (86° C.), dimethoxymethan (42.3° C.), diethoxymethan (88° C.), tetrahydrofuran (66° C.), (+/−)-tetrahydro-2-methylfuran (80° C.), tetrahydro-2,5-dimethylfuran (90-92° C.), tetrahydropyran (88° C.).

Preferably, the ether has a boiling point in the range of 50° C. to 80° C. under a pressure of 1 bar.

Examples of preferred ethers are methyl tert.-butyl ether (MTBE), ethyl tert.-butyl ether (ETBE), and diisopropyl ether. The most preferred ether is methyl tert.-butyl ether (MTBE).

The recrystallisation solvent contains at least 50%, preferably at least 70% and in particular at least 90% of one or more ethers, in particular of one or more ethers having a boiling point in the range of 40° C. to 100° C. under a pressure of 1 bar, especially of one or more of the ethers specifically identified above. These % values are intended to designate wt %, based on the total weight of the solvent.

While other solvents may be present besides the one or more ethers, the recrystallisation solvent contains less than 5%, preferably less than 2%, more preferably less than 1%, of aliphatic hydrocarbons as anti-solvents and in particular contains no aliphatic hydrocarbons. Preferably, these limitations apply to any hydrocarbon.

The above % values are intended to designate wt %, based on the total weight of the recrystallisation solvent, including hydrocarbon anti-solvents.

Further, preferably, while other solvents may be present besides the one or more ethers, the recrystallisation solvent contains less than 5%, preferably less than 2%, more preferably less than 1%, of any anti-solvents and in particular contains no anti-solvent. These % values are intended to designate wt %, based on the total weight of the recrystallisation solvent, including anti-solvents.

If more than one anti-solvent is present, the limitations set out above for the anti-solvent relate to the total amount of all anti-solvents present.

Minor amounts of certain anti-solvents may be present in commercially available solvents, such as commercially available ethers.

Preferably no anti-solvent is added in the method of the invention.

The term anti-solvent as used herein refers to a liquid in which the solubility of 5-MeO-DMT at any temperature occurring during the recrystallisation is less than 20% the solubility in the ether used, or, in case that more than one ether is used, in the combination of ethers present in the dissolution solvent. The solubility is expressed in mg/mL.

Typical anti-solvents include hydrocarbons, such as aliphatic hydrocarbons, like petrol ether, heptanes, hexanes; and water.

In a preferred embodiment, the recrystallisation solvent contains at least 50%, preferably at least 70% and in particular at least 90% of MTBE. In a specific embodiment, the recrystallisation solvent contains at least 98% MTBE. The % values are intended to designate wt %, based on the total weight of the solvent.

In a further preferred embodiment, 5-MeO-DMT is purified by recrystallisation in 98 wt % MTBE without adding a hydrocarbon as an anti-solvent.

The method of the invention involves dissolving crude 5-MeO-DMT in the recrystallisation solvent. The 5-MeO-DMT will be dissolved in the recrystallisation solvent at a dissolution temperature above room temperature up to the boiling point of the solvent.

Room temperature means a temperature in the range of 20 to 25° C. Above room temperature means above 25° C., below room temperature below 20° C.

In one embodiment, the 5-MeO-DMT will be dissolved in the recrystallisation solvent at a dissolution temperature in the range of 35 to 40° C.

The amount of recrystallisation solvent used is an amount sufficient to achieve complete dissolution at the dissolution temperature.

For instance, an at least 25% saturated solution is achieved. In one embodiment, the amount is sufficient to achieve a saturated solution at the dissolution temperature.

Preferably an at least 50% and at most 90% saturated solution is achieved.

After dissolution has occurred, the solution will be cooled to a temperature below room temperature (i.e., below 20° C.) to allow crystallisation of the 5-MeO-DMT. A suitable temperature range is 0 to 20° C. In one embodiment, the temperature is in the range of 7 to 12° C.

The product obtained can be recovered by filtration.

The recrystallisation may be repeated one or more times to improve purity.

The purification according to the present invention preferably does not involve a column chromatography step. Further, the purification according to the present invention does preferably not involve a step of distillation of 5-MeO-DMT.

From the product recovered residual solvent can, at least in part, be removed under reduced pressure. The weight amount of solvent in the product is NMT 5000 ppm, preferably NMT 2500 ppm, more preferably NMT 500 ppm and in particular NMT 100 ppm.

The method of the invention allows reducing the amount of impurities. It in particular allows reducing the total amount of impurities in a 5-MeO-DMT sample to below 0.5% and reducing the amount of each individual impurity in a 5-MeO-DMT sample to below 0.1%. These % values are area % determined by HPLC as indicated above. At the same time, low amounts of residual solvent can be achieved.

Thus, according to one aspect of the invention, 5-MeO-DMT is provided in a form containing a total amount of impurities of below 0.5 area %, wherein the amount of each individual impurity is below 0.1 area %. The amounts of impurities are determined by HPLC carried out with an octadecyl carbon chain-bonded silica (C18) column and a mixed solvent based on 0.013M ammonium acetate in water and acetonitrile as eluent, using UV detection at 227 nm.

5-MeO-DMT is provided in a form preferably having a residual solvent content of not more than 5000 ppm, preferably not more than 2500 ppm, more preferably not more than 500 ppm and in particular not more than 100 ppm.

The following examples are intended to further illustrate the invention.

### EXAMPLES

### Example 1—Analytical Method

The purity of 5-MeO-DMT is determined by HPLC.

### Method Parameters

Instrument: A suitable HPLC system equipped with UV detection, linked to the laboratory data handling system
Column: ACE C18 (150×4.6×3 μm)
Injection Volume: 5 □l
Flow Rate: 0.75 ml/minute
Detector: UV at 227 nm
Run Time: 25 minutes
Column Temperature: 30° C.
Diluent: Methanol
Mobile Phase A: 0.013M Ammonium acetate in water
Mobile Phase B: Acetonitrile

Gradient:

| Time (minutes) | Vol.-% Mobile Phase A | Vol.-% Mobile Phase B |
|---|---|---|
| 0.0 | 80 | 20 |
| 18.0 | 26 | 74 |
| 20.0 | 26 | 74 |
| 20.1 | 80 | 20 |
| 25.0 | 80 | 20 |

The typical retention time of 5-MeO-DMT is 5.5 min.

### Sample Preparation (in Duplicate)

Accurately weigh 12-18 mg of sample into 100 ml volumetric flask and make to volume with methanol. Mix well.

### Validation of Analytical Procedures

The HPLC method used was tested for linearity and precision. IR, NMR, GC-Headspace, KF and ICP-MS are common techniques, being applied according to routine procedures. Based on the results, the methods are considered as fit for purpose.

### Linearity of the HPLC Method

A stock solution of 5-MeO-DMT was prepared in methanol. A nominal concentration of 0.15 mg/ml was taken.

| | Actual | | | | |
|---|---|---|---|---|---|
| | concentration | Peak Area | | | |
| % Nominal | (mg/ml) | Injection 1 | Injection 2 | mean | % RD |
| 150 | 0.226 | 132.511 | 134.435 | 133.473 | 1.4 |
| 125 | 0.181 | 109.305 | 108.094 | 108.700 | −1.1 |
| 100 | 0.151 | 91.466 | 92.675 | 92.070 | 1.3 |
| 80 | 0.121 | 73.543 | 72.295 | 72.919 | −1.7 |
| 50 | 0.075 | 46.871 | 46.891 | 46.881 | 0.0 |
| 25 | 0.038 | 23.965 | 24.056 | 24.011 | 0.4 |
| 10 | 0.015 | 9.675 | 9.706 | 9.690 | 0.3 |
| 5 | 0.008 | 4.670 | 4.694 | 4.682 | 0.5 |
| 1 | 0.000 | 0.982 | 0.989 | 0.985 | 0.7 |
| 0.1 | 0.002 | 0.468 | 0.472 | 0.470 | 0.8 |
| 0.01 | 0.000 | 0.097 | 0.095 | 0.096 | −2.1 |

All duplicate injections were within +2%.

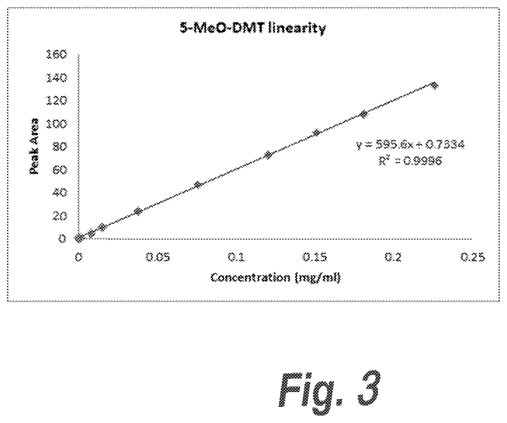

Y intercept % at nominal concentration was determined to be 0.8%. Method is deemed linear, as seen in FIG. 3.

### Precision of the HPLC Method

Six sample solutions were prepared at nominal concentration (12-18 mg in 100 ml methanol). The purity results were as follows:

| Precision | Purity (% area) |
|---|---|
| 1 | 99.21 |
| 2 | 99.02 |
| 3 | 99.18 |
| 4 | 99.21 |
| 5 | 99.17 |
| 6 | 99.17 |
| Average | 99.16 |
| SD | 0.07 |
| RSD (%) | 0.07 |

Acceptance criteria for purity values across the six samples would be 1% RSD, the actual reading was 0.07%. Therefore, the analytical method is considered to exhibit adequate precision.

### Example 2—Solvent Screen

A solubility screen of 5-MeO-DMT was performed on a number of solvents at both room temperature and reflux.

The screen involved adding a solvent to 200 mg of 5-MeO-DMT until complete dissolution was obtained, unless the compound had such a low solubility in the solvent that no complete dissolution of the sample was achieved in 4 ml solvent (solubility less than 0.05 mg/ml).

The results of the solubility screen are shown in the table below.

| | 5-MeO-DMT Solubility | | | |
|---|---|---|---|---|
| | Ambient | Ambient | Reflux | Reflux |
| Solvent | (g/mL) | (vols.) | (g/mL) | (vols.) |
| Acetone | 0.53 | 1.9 | — | — |
| Ethanol | 0.34 | 2.9 | — | — |
| Ethyl Acetate | 0.26 | 3.9 | — | — |
| 2-Methyltetrahydrofuran | 0.25 | 4.0 | — | — |
| (2-MeTHF) | | | | |
| Isopropyl acetate | 0.21 | 4.8 | — | — |
| (IPAc) | | | | |
| Toluene | 0.2 | 5.1 | — | — |
| Isopropanol (IPA) | 0.18 | 5.6 | — | — |
| Methyl tert-butyl | <0.05 | >20 | 0.42 | 2.4 |
| ether (MTBE) | | | | |
| Heptanes | <0.05 | >20 | <0.05 | >20 |
| Water | <0.05 | >20 | <0.05 | >20 |

The results from the solubility screen indicated that 5-MeO-DMT was very soluble in a number of solvents at room temperature. Poor solubility was observed in water and heptanes.

5-MeO-DMT was not observed to be soluble in MTBE at room temperature but was very soluble when the temperature of the solvent was increased. However, MTBE was not considered a suitable recrystallisation solvent as upon cooling of the hot MTBE solution to room temperature no crystallisation was observed.

### Example 3—Initial Attempts at Recrystallisation

Following the results of the solubility study a number of experiments were performed with the aim of recrystallising 5-MeO-DMT from a mixture of isopropanol (IPA) and either heptanes or water. The aim was to dissolve 5-MeO-DMT in IPA before adding water or heptanes as the anti-solvent to afford precipitation/crystallisation. IPA was used in order to maximise recovery of the recystallised 5-MeO-DMT as although 5-MeO-DMT was found to be very soluble in IPA at room temperature, 5-MeO-DMT was less soluble in IPA than in the other solvents evaluated.

In the first small scale experiment 5-MeO-DMT (200 mg) was dissolved in the minimum amount of IPA (0.3 mL, 1.5 volumes) required to achieve complete dissolution at 80° C. After cooling the solution to room temperature no crystallisation was observed, however. Therefore, heptanes were added dropwise with the aim of precipitating 5-MeO-DMT from solution. Unfortunately, upon the addition of heptanes 5-MeO-DMT was found to oil rather than precipitate. Oiling also occurred when the experiment was repeated using water as the anti-solvent in place of heptanes.

In a second attempt at crystallisation from an IPA and water or heptanes mixture the solvent additions were reversed in an attempt to stop oiling. 9.5 volumes (1.9 mL) of heptanes were charged to 5-MeO-DMT (200 mg) before the resulting slurry was warmed to 80° C. IPA (2.1 volumes, 0.4 mL) was charged dropwise until complete dissolution was observed. Upon cooling to room temperature, however, the material did not crystallise. An attempt with water (9.5 volumes, 1.9 mL) and IPA (5.3 volumes, 1 mL) also resulted in a lack of crystallisation upon cooling to room temperature.

To investigate whether crystallisation could be achieved using a reduced volume of the solvent mixtures a 17% IPA in heptanes solution (9.4 volumes, 1.9 mL) was added dropwise to 5-MeO-DMT (200 mg), at 80° C., until all material was completely dissolved. Unfortunately, after cooling, oiling was observed. Oiling also occurred when a 34% IPA in water (10.7 volumes) was added to 5-MeO-DMT (200 mg) at 80° C.

The previously described experiments utilising the 17% IPA in heptanes and 34% IPA in water solutions were repeated at 60° C. Unfortunately, in both cases oiling was observed upon cooling.

A further final small-scale experiment using a solvent in which 5-MeO-DMT has greater solubility was carried out. Ethanol (0.5 volumes, 0.1 mL) was charged to 5-MeO-DMT (200 mg) at 60° C. before water was added dropwise. However, upon addition of water to the batch oiling of 5-MeO-DMT was observed.

Table 2 below displays a summary of the small scale recrystallisation trials described above.

| | 5-MeO-DMT | | Anti- | |
|---|---|---|---|---|
| Experiment | charge | Solvent | solvent | Comments |
| IPA added until dissolution | 200 mg | IPA | Heptanes | Oiling out during dropwise |
| achieved at 80° C. | | | | addition of heptanes |
| Anti-solvent charged once | 200 mg | IPA | Water | Oiling out during dropwise |
| dissolution achieved | | | | addition of water |
| IPA charged to slurry of 5- | 200 mg | IPA | Heptanes | Complete dissolution |
| MeO-DMT in anti-solvent at | | | | achieved but no |
| 80° C. to achieve complete | | | | crystallisation upon cooling |
| dissolution | 200 mg | IPA | Water | Complete dissolution |
| | | | | achieved but no |
| | | | | crystallisation upon cooling |
| Mixture of IPA in anti-solvent | 200 mg | IPA | Heptanes | Complete dissolution |
| charged to 5-MeO-DMT at | | | | achieved but oiling out upon |
| 80° C. to achieve complete | | | | cooling |
| dissolution | 200 mg | IPA | Water | Complete dissolution |
| | | | | achieved but oiling out upon |
| | | | | cooling |
| Mixture of IPA in anti-solvent | 200 mg | IPA | Heptanes | Complete dissolution |
| charged to 5-MeO-DMT at | | | | achieved but oiling out upon |
| 60° C. to achieve complete | | | | cooling |
| dissolution | 200 mg | IPA | Water | Complete dissolution |
| | | | | achieved but oiling out upon |
| | | | | cooling |
| Ethanol added until dissolution | 200 mg | Ethanol | Water | Oiling out during dropwise |
| achieved at 60° C. Anti- | | | | addition of water |
| solvent charged once | | | | |
| dissolution achieved | | | | |

### Example 4—Crystallisation after Heating and Oiling

In an attempt to avoid 5-MeO-DMT oiling out during crystallisation a larger scale experiment was performed. With the increased scale, greater control over the anti-solvent charge was expected, allowing more careful addition, potential determination of the cloud point and the avoidance of oiling. 5-MeO-DMT (2.0 g) was warmed to 60° C. before being dissolved in the minimum amount of IPA (0.8 mL, 0.4 volumes) required to achieve complete dissolution. After cooling to room temperature no crystallisation was observed and so the batch was warmed back to 60° C. Water was added dropwise until the solution became cloudy. Unfortunately, no cloud point was determined because of 5-MeO-DMT oiling from solution without the solution becoming cloudy.

In order to determine whether it was possible to still crystallise 5-MeO-DMT, despite it oiling from solution, the solvent was removed by evaporation to yield an orange oil. After cooling the oil in a fridge overnight an orange crystalline solid was obtained. This result indicated that although crystallisation of 5-MeO-DMT was slow it was still possible to crystallise 5-MeO-DMT even after heating and oiling.

### Example 5—Larger Scale Crystallisation Attempts Using MTBE/Heptanes

Due to the issues encountered when using a solvent mixture of either IPA or ethanol in heptanes or water, a solvent system utilising MTBE was reinvestigated.

Previously crystallisation from MTBE was not observed following cooling to room temperature. Therefore, in the second attempt at crystallisation, from MTBE, the solution was further cooled to 0 to 5° C.

MTBE (1 mL, 0.5 volumes) was charged to the crystalline solid at 45 to 50° C. until complete dissolution was achieved. This MTBE charge was lower than the 2.4 volumes stated previously and is likely due to the small scale that the solvent screen was performed at.

Following dissolution, the batch was cooled to 0 to 5° C. over 40 minutes to yield a very thick viscous solution that did afford crystallisation.

The batch was warmed back to 45 to 50° C. and then cooled to room temperature over 1 hour. After stirring at room temperature for 30 minutes the batch became a very thick viscous solution and so a further 0.4 mL (0.2 volumes) of MTBE was charged before the batch was stirred for a further 15 minutes. Following the stirring a thin slurry was obtained.

Heptanes (0.7 mL, 0.35 volumes) were charged dropwise and then the batch was stirred for an additional 15 minutes resulting in a thicker slurry. The batch was cooled to 0 to 5° C. and heptanes (0.7 mL, 0.35 volumes) were subsequently charged dropwise. Once filtered the isolated solids were washed with cold heptanes (2×2 mL) before being dried under vacuum overnight to yield a pale orange solid. The isolated solid (1.44 g, 72% recovery) was analysed by HPLC for purity.

Experimental conditions are summarised in Table 3. The results of the analysis are shown in Table 4.

### Example 6—Crystallisation Utilising MTBE/Heptanes

A second experiment utilising MTBE/heptanes was performed. 5-MeO-DMT (2.0 g) was dissolved in MTBE (1.4 mL, 0.7 volumes) at 45 to 50° C. before being cooled to room temperature over 70 minutes. Upon crystallisation stirring stopped and so the batch was warmed back to 45 to 50° C. and additional MTBE (1 mL, 0.5 volumes) was charged. After cooling back to room temperature and stirring out the resulting slurry was diluted with heptanes (1 mL, 0.5 volumes). Upon heptanes addition a large amount of material was observed to coat the vessel wall and so to try and remove this material a further amount MTBE (1 mL, 0.5 volumes) was charged. After stirring for 90 minutes at room temperature the batch was filtered then washed sequentially with MTBE: heptanes (2:1, 1 mL, 0.5 volumes) and heptanes (1 mL, 0.5 volumes). After drying a pale orange solid was obtained in 1.13 g (57% recovery). The isolated material was analysed for purity by HPLC.

Experimental conditions are summarised in Table 3. The results of the analysis are shown in Table 4.

### Example 7—Crystallisation Utilising MTBE/Heptanes

5-MeO-DMT (2.0 g) was dissolved in MTBE (2 mL, 1.0 volume) at 45 to 50° C. before being cooled to room temperature over 1 hour. After stirring out at room temperature for 1.5 hours the resulting slurry was diluted with MTBE (2×1 mL, 1.0 volume) over 20 minutes. Heptanes (1 mL, 0.5 volumes) were then charged over 5 minutes before the batch was stirred at room temperature for 2 hours. After cooling to 8 to 12° C. over 10 minutes the batch was stirred for 15 minutes at 8 to 12° C. before being diluted with more heptanes (1 mL, 0.5 volumes). After an additional 10 minutes stir out at 8 to 12° C. the batch was cooled to 0 to 5° C. over 10 minutes then stirred out for a further 15 minutes. Heptanes (2 mL, 1.0 volume) were then charged over 5 minutes before the batch was stirred at 0 to 5° C. for 1 hour. The batch was filtered and washed with MTBE: heptanes (1:1, 2 mL, 1.0 volume). After drying a pale orange solid was obtained in 1.37 g (69% recovery). The isolated solid was analysed for purity by HPLC.

Experimental conditions are summarised in Table 3. The results of the analysis are shown in Table 4.

### Example 8—Crystallisation from MTBE

In order to try and further increase the purity of the recystallised material an experiment was performed using only MTBE as the solvent. 5-MeO-DMT (2.0 g) was dissolved in MTBE (4 mL, 2.0 volumes) at 35 to 40° C. before being cooled to room temperature over 30 minutes. After stirring at room temperature for 50 minutes no crystallisation was observed, therefore, the batch temperature was decreased to 7 to 12° C. over 30 minutes. After stirring at 7 to 12° C. for 10 minutes crystallisation occurred. The batch was subsequently filtered following a 1 hour stir out at 7 to12° C. After washing with MTBE (1 mL, 0.5 volumes), at 7 to 12° C., the batch was pulled dry under vacuum for 3.5 hours to yield a pale orange solid in 1.02 g (50% recovery). The isolated solid was analysed for purity by HPLC.

Experimental conditions are summarised in Table 3. The results of the analysis are shown in Table 4.

Table 3 below displays a summary of the conditions of the larger scale attempts at recrystallisation of 5-MeO-DMT.

| | Example 5 | Example 6 | Example 7 | Example 8 |
|---|---|---|---|---|
| Input (g) | 2.00 | 2.00 | 2.00 | 2.0 |
| MTBE:heptanes ratio | 1:1 | 4:1 | 1:1 | No heptanes |
| Total solvent | 2.8 | 4.4 | 8.0 | 4.0 |
| volume (mL) | | | | |
| Total solvent | 1.2 | 2.2 | 4.0 | 2.0 |
| volume (vols.) | | | | |
| Isolation | 0-5 | 20-25 | 0-5 | 7-12 |
| temperature (° C.) | | | | |
| Recovery (g) | 1.44 | 1.13 | 1.37 | 1.02 |
| Recovery (%) | 72 | 57 | 69 | 50 |
| Purity (% area) | 99.61 | 99.58 | 99.45 | 99.74 |

Table 4 below displays impurity profiles of materials isolated from recrystallisation experiments.

| | | HPLC Purity (area %) | | | | |
|---|---|---|---|---|---|---|
| Impurity Profile | | Raw | | | | |
| | RRT | Material | Example 5 | Example 6 | Example 7 | Example 8 |
| | 0.87 | 0.07 | — | 0.02 | 0.02 | 0.06 |
| | 0.90 | 0.04 | 0.08 | 0.05 | 0.06 | 0.02 |
| | 0.92 | 0.03 | 0.02 | 0.02 | 0.02 | — |
| 5-MeO-DMT | 1.00 | 99.21 | 99.61 | 99.58 | 99.45 | 99.74 |
| | 1.18 | 0.13 | 0.03 | 0.11 | 0.14 | 0.04 |
| | 1.24 | 0.15 | 0.06 | 0.07 | 0.05 | 0.02 |
| | 1.28 | 0.02 | — | — | 0.01 | <0.01 |
| | 1.64 | — | — | — | 0.01 | 0.02 |
| | 1.67 | — | — | — | — | <0.01 |
| | 1.72 | — | — | — | 0.02 | — |
| | 1.96 | 0.02 | — | — | 0.01 | — |
| | 2.08 | — | — | — | 0.01 | — |
| | 2.11 | — | — | — | 0.01 | — |
| | 2.34 | 0.03 | 0.02 | 0.03 | 0.03 | — |
| | 2.38 | 0.29 | 0.19 | 0.13 | 0.14 | 0.08 |
| | 2.42 | — | — | — | 0.01 | — |
| | 2.61 | — | — | — | <0.01 | — |
| | 2.76 | 0.01 | — | — | 0.01 | — |
| | 2.82 | — | — | — | 0.01 | — |
| | 2.90 | — | — | — | <0.01 | — |

A summary of the conditions used in the each of the larger scale recrystallisation experiments is shown in Table 3. The results from the analysis indicated that in all cases the overall purity of the material was increased and the impurity at RRT 1.24 was purged to below 0.10%. The impurity at RRT 2.38 was also reduced in all three experiments however but only below the target of NMT 0.10% in the experiment according to Example 8. The impurity at RRT 1.18 was only purged using the experimental conditions according to Example 5 and according to Example 8. Based upon these results it appears the most suitable conditions for achieving material in which all impurities are below 0.10% are from the experiment according to Example 8. Solvent analysis of sample according to Example 8 indicated an MTBE level of 17 ppm against an expected limit of NMT 5000 ppm.

1. Method of purifying 5-methoxy-N,N-dimethyltryptamine (5-MeO-DMT) comprising
dissolving crude 5-MeO-DMT in a solvent at a temperature above room temperature
cooling the obtained solution to a temperature below room temperature to precipitate solid 5-MeO-DMT
separating the solid 5-MeO-DMT from the remaining solution
removing solvent from the crystalline 5-MeO-DMT
wherein the solvent comprises one or more ethers and less than 5 wt % of aliphatic hydrocarbons as anti-solvents.

2. Method according to claim 1, wherein the one or more ethers have boiling points in the range of 40° C. to 100° C. under a pressure of 1 bar.

3. Method according claim 1 or 2, wherein the one or more ethers are selected from methyl tert.-butyl ether (MTBE), ethyl tert.-butyl ether (ETBE), and diisopropyl ether.

4. Method according to any of the preceding claims, wherein the recrystallisation solvent contains at least 50 wt %, preferably at least 70 wt % and in particular at least 90 wt % of one or more ethers.

5. Method according to any of the preceding claims, wherein the recrystallisation solvent contains less than 2 wt % aliphatic hydrocarbons.

6. Method according to claim 5, wherein the recrystallisation solvent contains less than 1 wt % aliphatic hydrocarbons.

7. Method according to any of the preceding claims, wherein the recrystallisation solvent contains at least 98 wt % MTBE.

8. Method according to any of the preceding claims, wherein the solvent comprises less than 5 wt % in total of any anti-solvents, wherein anti-solvent means a liquid in which the solubility of 5-MeO-DMT at any temperature occurring during the recrystallisation is less than 20% the solubility in the ether used, or, in case that more than one ether is used, in the combination of ethers present in the dissolution solvent.

9. Method according to claim 8, wherein the recrystallisation solvent contains less than 2 wt % of anti-solvents.

10. Method according to claim 9, wherein the recrystallisation solvent contains less than 1 wt % of anti-solvents.

11. Method according to any of the preceding claims, wherein the solution is cooled to a temperature in the range of 0 to 20° C.

12. Method according to claim 11, wherein the temperature is in the range of 7 to 12° C.

13. Method according to any of the preceding claims additionally comprising a step of reducing the amount of remaining recrystallisation solvent.

14. Method according to any of the preceding claims, wherein the total amount of impurities is reduced to below 0.5 area % as determined by HPLC using UV detection at 227 nm and/or wherein the amount of each individual impurity is reduced to below 0.1 area % as determined by HPLC using UV detection at 227 nm.

15. Method according to claim 14, wherein the HPLC is carried out with an octadecyl carbon chain-bonded silica (C18) column and a mixed solvent based on 0.013M ammonium acetate in water and acetonitrile as eluent.

16. Method according to any of claims 13 to 15, wherein the amount of residual solvent in the product is not more than 5000 ppm.

17. 5-Methoxy-N,N-dimethyltryptamine (5-MeO-DMT), wherein the total amount of impurities is below 0.5 area % and wherein the amount of each individual impurity is below 0.1 area %, wherein the amounts of impurities are determined by HPLC carried out with an octadecyl carbon chain-bonded silica (C18) column and a mixed solvent based on 0.013M ammonium acetate in water and acetonitrile as eluent, using UV detection at 227 nm.

18. 5-MeO-DMT according to claim 17, wherein in addition the amount of residual solvent is not more than 5000 ppm.
